(19) United States
(12) Patent Application Publication
(10) Pub. No.: US 2019/0352297 A1
(43) Pub. Date: Nov. 21, 2019

(54) SELECTIVE AURORA A KINASE INHIBITORS

(71) Applicant: UNIVERSITAT BERN, Bern (CH)

(72) Inventors: Jean-Louis REYMOND, Bulle (CH); Falco KILCHMANN, Danikon (CH)

(73) Assignee: UNIVERSITAT BERN, Bern (CH)

(21) Appl. No.: 16/476,295

(22) PCT Filed: Jan. 6, 2017
(86) PCT No.: PCT/EP2017/050283

§ 371 Date: Jul. 7, 2019

(51) Int. Cl. C07D 417/06; C07D 417/14

(52) U.S. Cl. CPC C07D 417/06; C07D 417/14

(57) ABSTRACT

The invention relates to a compound

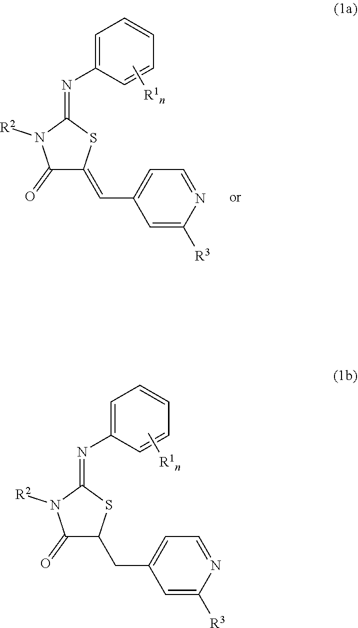

wherein R¹ is selected from —I, —Br, —Cl, —F, C₁-C₆-alkyl, —O(CH₂)ₘCH₃, —(CH₂)ₘOCH₃, C₁-C₆-haloalkyl, a cycloalkyl, a heterocycle, a aryl or heteroaryl, wherein R² is selected from H, C₁-C₆-alkyl, C₁-C₆-haloalkyl, C₃-C₆-cycloalkyl, —CH₂—(C₃-C₆-cycloalkyl), or —(CH₂)ᵣOCH₃, wherein R³ is —NH₂, —NH—R⁴, —NHC(═O)—R⁴, —NHC(═O)NH—R⁴, —NHC(═S)—R⁴ or —NHC(═S)NH—R⁴, wherein R⁴ is selected from a cycloalkyl, a heterocycle, a aryl or a heteroaryl, or -L-R⁵, wherein L is selected from C₁-C₅-alkyl, a cycloalkyl, a heterocycle, a aryl, or a heteroaryl, and R⁵ is selected from —OH, —CH₂OH, —NH₂, —COOH, —CONH₂, —CONH—R⁶ or carboxylic acid isosteres, wherein R⁶ is selected from C₁-C₄-alkyl, with n, m and r being 0, 1, 2, 3, 4 or 5 and their use.

### FIELD OF THE INVENTION

The present invention relates to a class of thiazolidinone derivatives as selective inhibitors of Aurora A and their use in the treatment of cancer.

### BACKGROUND OF THE INVENTION

The inventors identified new and selective inhibitors of Aurora A, an evolutionary conserved serine/threonine kinase essential for proper progression through mitosis and one of the most intensively researched kinase targets due to its significance in cancer. Although many potent inhibitors of this kinase are known, most of these also inhibit Aurora B, a chromosomal passenger protein required notably for cytokinesis and which shares over 85% sequence homology with Aurora A in the kinase domain.

Alisertib (MLN8237), one of the most selective Aurora A inhibitors reported to date (de Groot et al., Front Oncol, 2015, 5, 285), is currently under advanced clinical investigation for the treatment of various solid and hematological malignancies. Interestingly, alisertib and its close relative MLN8054 (Hoar et al., Mol Cell Biol, 2007, 27, 4513-4525) belong to a scaffold that is unique among the 4,874 scaffolds, as defined by Bemis and Murcko (Bemis and Murcko, J Med Chem, 1996, 39, 2887-2893), occurring in 11,286 kinase inhibitors with potencies better than 50 nM listed in ChEMBL (Gaulton et al., Nucleic Acids Res, 2012, 40, D1100-D1107). Furthermore, the diversity of scaffolds is quite high among Aurora A kinase inhibitors, with 174 different scaffolds occurring among 329 inhibitors with potencies better than 50 nM listed in ChEMBL. This suggests that each inhibitor represents an already optimized compound for each scaffold, and that discovering further selective inhibitors for this particular kinase requires identifying different scaffolds.

### DESCRIPTION

The inventors provide herein compounds that specifically inhibit Aurora A. These compounds are for use as a medicament and useful for the treatment of cancer.

A first aspect of the invention relates to a compound comprising the general formula (1a) or (1b), in particular (1a),

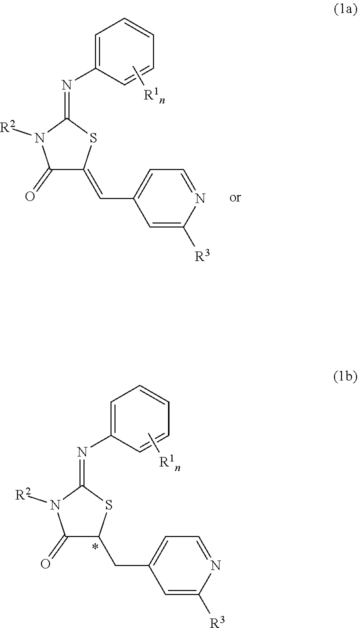

wherein
R¹ is selected from —I, —Br, —Cl, —F, C₁-C₆-alkyl, —O(CH₂)ₘCH₃—(CH₂)ₘOCH₃, C₁-C₆-haloalkyl, a substituted or unsubstituted cycloalkyl, a substituted or unsubstituted heterocycle, a substituted or unsubstituted aryl or a substituted or unsubstituted heteroaryl, wherein
   n is 0, 1, 2, 3, 4 or 5 and
   m is 0, 1, 2, 3, 4 or 5
   R² is selected from H, C₁-C₆-alkyl, or C₁-C₆-haloalkyl, C₃-C₆-cycloalkyl, —CH₂—(C₃-C₆-cycloalkyl), or —(CH₂)ᵣOCH₃, wherein
   r is 1, 2, 3, 4 or 5
R³ is
   NH₂, —NH—R⁴, —NHC(═O)—R⁴, —NHC(═O)NH—R⁴, —NHC(═S)—R⁴ or —NHC(═S)NH—R⁴, wherein
      R⁴ is selected from
      a substituted or unsubstituted cycloalkyl, a substituted or unsubstituted heterocycle, a substituted or unsubstituted aryl or a substituted or unsubstituted heteroaryl, or
      L-R⁵, wherein
         L is selected from
         C₁-C₅-alkyl,
         a substituted or unsubstituted cycloalkyl, a substituted or unsubstituted heterocycle, a substituted or unsubstituted aryl, or a substituted or unsubstituted heteroaryl, and
         R⁵ is selected from —OH, —CH₂OH, —NH₂, —COOH, —CONH₂, CONH—R⁶ or carboxylic acid isosteres, wherein
         R⁶ is selected from C₁-C₄-alkyl, in particular C₁-C₂-alkyl.

The compounds disclosed herein specifically inhibit Aurora A by binding to its catalytic pocket.

Inhibitors of formula (1a) or (1b) induce an inactive DFG-motif conformation that comprises distortion of the R-spine, an outward displacement of the aC helix, absence of the salt-bridge between Lys162 and Glu181 and disruption of the hydrogen bond network between Asp156, Thr292 and Lys258 of Aurora A. As the activator protein TPX2 induces the formation of the salt-bridge between Lys162 and Glu181, inhibitors of formula (1a) or (1b) do not only prevent ATP binding but also Aurora A activation by TPX2. Optimal binding to Aurora A is achieved if both exocyclic bonds of the thiazolidinone in compounds of formula (1a) are in Z-configuration. The inhibitors of formula (1a) or (1b) bind to the ATP binding pocket of the ATP binding region of Aurora A and form hydrophobic contacts with Leu139, Val14, Ala160 and Leu263. The phenyl moiety of these inhibitors binds to the hydrophobic back pocket of Aurora A by forming hydrophobic contacts with Leu194, Arg195, Leu196, Leu210 and Phe275. Inhibitor binding induces an upward rotation of Phe275 of Aurora A that interacts with the phenyl ring of a compound of formula (1a) or (1b) upon binding. The 4-pyridine ring of an inhibitor of formula (1a) or (1b) binds to the hinge region of Aurora A by forming a hydrogen bond with Ala213.

The compounds of formula 1(b) are racemic mixtures. The stereocenter is marked by a star.

In some embodiments, R¹ is selected from C₁-C₆-alkyl, —I, —Br, —Cl, —F, —O(CH₂)ₘCH₃, —(CH₂)ₘOCH₃, cycloalkyl, in particular C₃-C₆-cycloalkyl, more particularly C₆-cycloalkyl or C₁-C₆-haloalkyl, wherein m is 0, 1, 2, 3, 4 or 5.

In some embodiments, R¹ is selected from C₁-C₆-alkyl, —O(CH₂)ₘCH₃, —I, —Br, —Cl, —F or C₁-C₆-haloalkyl, wherein m is 0, 1, 2, 3, 4 or 5.

In some embodiments, R¹ is selected from C₁-C₄-alkyl, C₁-C₄-haloalkyl, —O(CH₂)ₘCH₃, —I, —Br, —Cl or —F, wherein m is 0, 1, 2 or 3.

In some embodiments, R¹ is selected from C₁-C₃-alkyl, —CH₂CF₃, —CHFCF₃, —CF₂CF₃, —CHF₂, —CH₂F or —CF₃, —O(CH₂)ₘCH₃, —I, —Br, —Cl or —F, wherein m is 0, 1, 2 or 3.

In some embodiments, R¹ is selected from methyl, ethyl or isopropyl, —CH₂CF₃ or CF₃, —O(CH₂)ₘCH₃, —I, —Br, —Cl or —F, wherein m is 0, 1, 2 or 3.

In some embodiments, R¹ is selected from C₁-C₄-alkyl, C₁-C₄-haloalkyl, —O(CH₂)ₘCH₃, —I, —Br, —Cl or —F, wherein m is 0, 1, 2 or 3.

In some embodiments, R¹ is selected from C₁-C₃-alkyl, —CH₂CF₃, —CHFCF₃, —CF₂CF₃, —CHF₂, —CH₂F or —CF₃, —I, —Br, —Cl or —F.

In some embodiments, R¹ is selected from methyl, ethyl or isopropyl, —CH₂CF₃ or CF₃, —I, —Br, —Cl or —F.

In some embodiments, R¹ is selected from —Br, —CF₃ or ethyl.

In some embodiments, R¹ is ethyl.

In some embodiments, n of R¹ₙ is 0, 1 or 2, particularly 1.

In some embodiments, n of R¹ₙ is 1 and R¹ is a para-substitution.

R¹ is a substitute at the phenyl moiety of compound of formula (1a) or (1b) that binds to the hydrophobic back pocket of Aurora A. Therefore, suitable substituents comprise hydrophobic moieties such as alkyls, haloalkys or halogens. Potent inhibitors of formula (1a) or (1b) are characterized by a small alkyl substituent (R¹) in para position, for example 4-methyl or 4-ethyl.

In some embodiments, R² is selected from H, C₁-C₆-alkyl, or —(CH₂)ᵣOCH₃, wherein r is 1, 2, 3, 4 or 5.

In some embodiments, R² is selected from H, C₁-C₄-alkyl or —(CH₂)ᵣOCH₃, with r being 1, 2 or 3, wherein in particular r is 1 or 2.

In some embodiments, R² is selected from H, C₁-C₂-alkyl or —(CH₂)₂OCH₃.

In some embodiments R² is C₁-C₂-alkyl.

In some embodiments, R² is —CH₃.

The thiazolidinone moiety in compounds of formula (1a) or (1b) interacts with the ATP binding region of Aurora A. Substituents at the endocyclic nitrogen atom (R²) may form hydrophobic contacts with Val147 of Aurora A. Therefore, small hydrophobic substituents such as methyl or ethyl are required at this position.

In some embodiments, R³ is —NH₂, —NH—R⁴ or —NHC(═O)—R⁴.

In some embodiments, R³ is selected from —NH—R⁴ or —NHC(═O)—R⁴.

The 4-pyridine ring of an inhibitor of formula (1a) or (1b) binds to the hinge region of Aurora A by forming a hydrogen bond with Ala213. A second hydrogen bond may be formed if the 2-pyridyl position is substituted with a hydrogen bond acceptor such as a substituted or unsubstituted amino group.

In some embodiments, R⁴ is selected from a substituted or unsubstituted aryl or a substituted or unsubstituted heteroaryl, or -L-R⁵, wherein in particular R⁴ is -L-R⁵.

In some embodiments, R⁴ is selected from a substituted or unsubstituted C₆-aryl, a substituted or unsubstituted C₆-heteroaryl, or -L-R⁵, wherein in particular R⁴ is -L-R⁵.

In some embodiments, R⁴ is phenyl, or 2-pyridyl, or -L-R⁵, wherein in particular R⁴ is -L-R⁵.

The binding of an inhibitor of formula (1a) or (1b) can be further enhanced by substituents at the amino group at the 2-pyridyl position. Such substituents, for example phenyl or pyridyl, may form hydrophobic contacts with Gly216 and Leu263 of Aurora A.

In some embodiments, R⁴ is -L-R⁵.

In some embodiments, L is selected from C₁-C₅-alkyl, in particular C₁-C₃-alkyl, a substituted or unsubstituted aryl, or a substituted or unsubstituted heteroaryl.

In some embodiments, L is selected from C₆-aryl, C₆-heteroaryl or C₁-C₅-alkyl, in particular C₁-C₃-alkyl.

In some embodiments, L is selected from pyridyl or C₁-C₅-alkyl, in particular C₁-C₃-alkyl.

In some embodiments, R⁵ is selected from —OH, —CH₂OH, —NH₂, —COOH, —CONH₂, CONH—R⁶ or carboxylic acid isosteres according to scheme (1), wherein R⁶ is selected from C₁-C₄-alkyl, in particular C₁-C₂-alkyl.

In some embodiments, R⁵ is selected from —CH₂OH, —NH₂, —CONH₂, —CONH—R⁶, —COOH, tetrazole, with R⁶ being selected from C₁-C₄-alkyl, in particular C₁-C₂-alkyl.

In some embodiments, R⁵ is selected from —CH₂OH, —NH₂, —CONH₂, —COOH, tetrazole.

In some embodiments, R⁵ is selected from —CH₂OH, —NH₂, —COOH, tetrazole.

In some embodiments, R⁵ is selected from —CH₂OH, —NH₂ or —COOH.

In some embodiments, R⁵ is COOH.

In some embodiments, R³ is —NHC(═O)-L-R⁵, wherein L is selected from C₁-C₅-alkyl, in particular C₁-C₃-alkyl, and R⁵ is selected from —COOH or —NH₂, in particular —COOH.

In some embodiments, R³ is —NH-L-R⁵, wherein L is a substituted or unsubstituted aryl, in particular a 6-membered substituted or unsubstituted aryl, more particularly phenyl, or a substituted or unsubstituted heteroaryl, in particular a 6-membered substituted or unsubstituted heteroaryl, more particularly 2-pyridyl, and R⁵ is selected from —COOH or —NH₂, in particular —COOH.

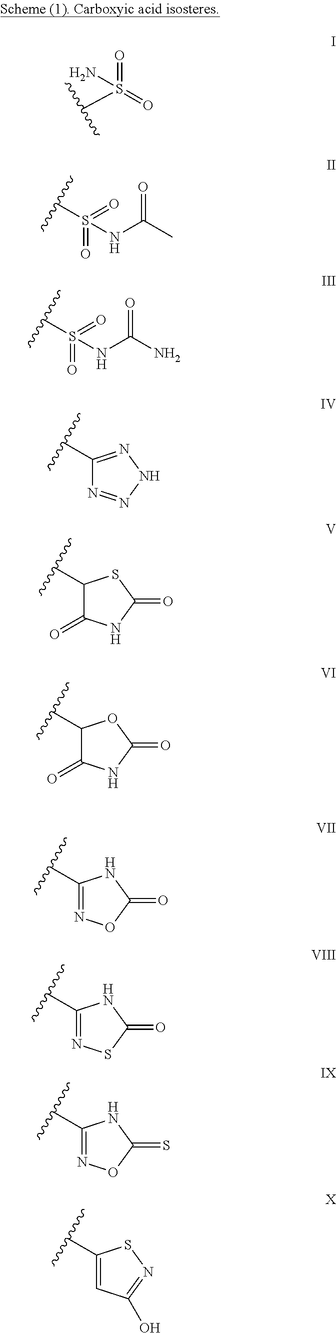

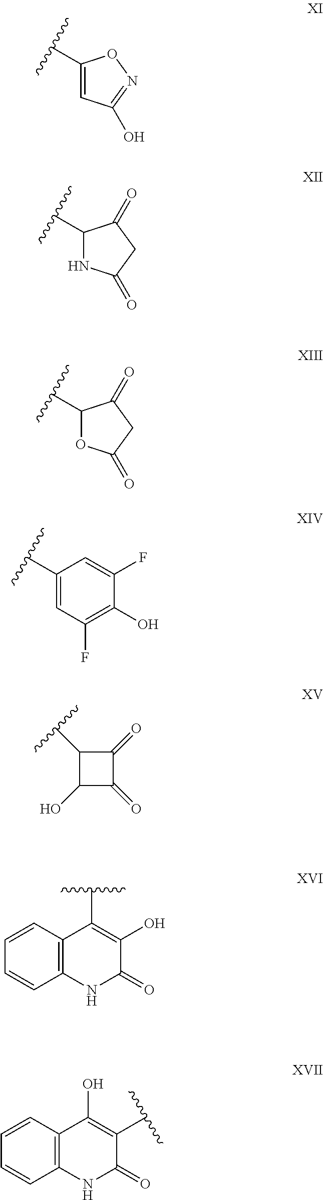

The most potent inhibitors of formula (1a) or (1b) are characterized by a linker L that can form hydrophobic contacts with Gly216 and Leu263 of Aurora A and an hydrogen bond acceptor such as a carboxylate group (R⁵) that may form hydrogen bonds with Arg137 of Aurora A.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is selected from a substituted or unsubstituted aryl or a substituted or unsubstituted heteroaryl or -L-R⁵, and L is selected from C₁-C₅-alkyl, in particular C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, and
R¹ is selected from —I, —Br, —Cl, —F, C₁-C₄-alkyl, or C₁-C₄-haloalkyl, and
R² is selected from H, C₁-C₄-alkyl or —(CH₂)ᵣOCH₃, with r being 1, 2 or 3, wherein in particular r is 2.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is selected from a substituted or unsubstituted aryl or a substituted or unsubstituted heteroaryl or -L-R⁵, and L is selected from C₁-C₅-alkyl, in particular C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, and
R¹ is selected from —Br, —CF₃ or C₁-C₃-alkyl, and
R² is selected from C₁-C₂-alkyl.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is selected from a substituted or unsubstituted aryl or a substituted or unsubstituted heteroaryl or -L-R⁵, and L is selected from C₁-C₅-alkyl, in particular C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, and
R¹ is ethyl, and
R² is —CH₃.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is -L-R⁵, and L is selected from C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, and
R¹ is selected from —I, —Br, —Cl, —F, C₁-C₄-alkyl, or C₁-C₄-haloalkyl, and
R² is selected from H, C₁-C₄-alkyl or —(CH₂)ᵣOCH₃, with r being 1, 2 or 3, wherein in particular r is 2.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is -L-R⁵, and L is selected from C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, and
R¹ is selected from —Br, —CF₃ or C₁-C₃-alkyl, and
R² is selected from C₁-C₂-alkyl.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is -L-R⁵, and L is selected from C₁-C₅-alkyl, in particular C₁-C₃-alkyl, a substituted or unsubstituted aryl, or a substituted or unsubstituted heteroaryl, and R⁵ is selected from —CH₂OH, —NH₂, —COOH, tetrazole, in particular —CH₂OH, —NH₂ or —COOH, more particularly COOH, R¹ is ethyl, and
R¹ is ethyl, and
R² is —CH₃.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is -L-R⁵, and L is selected from C₆-aryl or C₆-heteroaryl, or C₁-C₃-alkyl, and R⁵ is selected from —CH₂OH, —NH₂, —COOH, tetrazole, in particular —CH₂OH, —NH₂ or —COOH, more particularly —COOH, and
R¹ is selected from —Br, —CF₃ or C₁-C₃-alkyl, and
R² is selected from C₁-C₂-alkyl.

In some embodiments, R³ is selected from —NH₂, —NH—R⁴, —NHC(═O)—R⁴, —NHC(═O)NH—R⁴, —NHC(═S)—R⁴ or —NHC(═S)NH—R⁴, in particular from —NH—R⁴ or —NHC(═O)—R⁴, and
R⁴ is -L-R⁵, and L is selected from pyridyl, or C₁-C₃-alkyl, and R⁵ is selected from —CH₂OH, —NH₂, —COOH, tetrazole, in particular —CH₂OH, —NH₂ or —COOH, more particularly —COOH, and
R¹ is ethyl, and
R² is —CH₃.

A second aspect of the invention relates to a compound according to the first aspect of the invention for use as a medicament.

A third aspect of the invention relates to a compound according to the first aspect of the invention for use in the treatment of cancer.

A fourth aspect of the invention relates to a compound according to the first aspect of the invention for use as an inhibitor of Aurora A.

A fifth aspect relates to a method for treating or preventing a disease, comprising administrating a compound according to the first aspect of the invention to a patient in need thereof, in particular in a pharmaceutically effective amount, more particularly wherein said disease is cancer.

The compounds of the invention are selective inhibitors of Aurora A, an evolutionary conserved serine/threonine kinase essential for proper progression through mitosis. Due to its role in cell proliferation and elevated expression profile in many human cancers, Aurora A is an anti-cancer target. Binding of the compounds of the invention to Aurora A results in decreased autophosphorylation of Thr288 of Aurora A, which marks Aurora A kinase activity, defective chromosome alignment during metaphase and impaired Aurora A localization at the spindle microtubules.

In some embodiments, the compounds of the general formulas (1a) or (1b) may be isolated in form of salts, in particular in form of pharmaceutically acceptable salts. The same applies to all of the before mentioned embodiments. In some embodiments, the compounds of the general formulas (1a) or (1b) may be isolated in form of a tautomer, a hydrate or a solvate. Such salts are formed, for example, as acid addition salts, preferably with organic or inorganic acids, from compounds of the general formulas (1a) or (1b) with a basic nitrogen atom, in particular the pharmaceutically acceptable salts are formed in such a way. Suitable inorganic acids are, without being limited to, halogen acids, such as hydrochloric acid, sulfuric acid, or phosphoric acid and the like. Suitable organic acids are, without being limited to, carboxylic, phosphonic, sulfonic or sulfamic acids and the like. Such organic acids may be, without being limited to, acetic acid, glycolic acid, lactic acid, malic acid, tartaric acid, or citric acid. Salts may also be formed, for example, as salts with organic or inorganic bases, from compounds of the general formulas (1a) or (1b) with a nitrogen atom bearing an acidic hydrogen. Examples of suitable cations are—without being limited to—sodium, potassium, calcium or magnesium cations, or cations of organic nitrogen bases, e.g. protonated mono-, di- or tri-(2-hydroxethyl)amine.

In view of the close relationship between the novel compounds in their free form and those in the form of their salts, any reference to the free compounds hereinbefore and hereinafter is to be understood as referring also to the corresponding salts, as appropriate and expedient. The same applies to a hydrate or a solvate.

In some embodiments, the pharmaceutical preparation comprises at least one compound according to the invention as an active ingredient and at least one pharmaceutically acceptable carrier. In some embodiments, the pharmaceutical preparation comprises at least one compound according to the invention in its free form as an active ingredient. In some embodiments, the pharmaceutical preparation comprises at least one compound according to the invention in its free form as an active ingredient and at least one pharmaceutically acceptable carrier.

In some embodiments, the pharmaceutical preparation comprises at least one compound according to the invention in form of a salt, a tautomer, a pharmaceutically acceptable salt, a hydrate or a solvate. In some embodiments, the pharmaceutical preparation comprises at least one compound according to the invention in form of a salt, a tautomer, a pharmaceutically acceptable salt, a hydrate or a solvate and at least one pharmaceutically acceptable carrier.

Furthermore the invention relates to pharmaceutical preparations comprising at least one compound mentioned herein before as active ingredient, which can be used especially in the treatment of the diseases mentioned. The pharmaceutical preparations may be used in particular for a method for treatment of cancers.

### Terms and Definitions

In the context of the present specification, the term “R-spine” refers to four conserved hydrophobic amino acid residues that form a column in the active state of a kinase.

In the context of the present specification, the term “DFG-motif” refers to a conserved Asp-Phe-Gly motif at the N terminus of the activation loop of kinases. The kinase is catalytically inactive if the activation loop is flipped out relative to its conformation in the catalytically active state. The inactive conformation is referred to as “DFG-out conformation” whereas the active conformation is referred to as “DFG-in conformation”.

The term “substituted” refers to the addition of a substituent group to a parent moiety.

“Substituent groups” can be protected or unprotected and can be added to one available site or to many available sites in a parent moiety. Substituent groups may also be further substituted with other substituent groups and may be attached directly or by a linking group such as an alkyl, an amide or hydrocarbyl group to a parent moiety. “Substituent groups” amenable herein include, without limitation, halogen, oxygen, nitrogen, sulphur, hydroxyl, alkyl, alkenyl, alkynyl, acyl, carboxyl, aliphatic groups, alicyclic groups, alkoxy, substituted oxy, aryl, aralkyl, amino, imino, amido fluorinated compounds etc.

As used herein the term “alkyl,” refers to a saturated straight or branched hydrocarbon moiety containing in particular up to 6 carbon atoms. Examples of alkyl groups include, without limitation, methyl, ethyl, propyl, butyl, isopropyl, n-hexyl, and the like. Alkyl groups typically include from 1 to about 6 carbon atoms (C₁-C₆ alkyl).

As used herein the term “cycloalkyl” refers to an interconnected alkyl group forming a saturated or unsaturated (or partially unsaturated) ring or polyring structure containing 3 to 10, particularly 5 to 10 carbon atoms. Examples of cycloalkyl groups include, without limitation, cyclopropane, cyclopentane, cyclohexane, norbornane, decaline or adamantan (Tricyclo[3.3.1.1]decan), and the like. Cycloalkyl groups typically include from 5 to 10 carbon atoms (C₅-C₁₀ cycloalkyl), in particular 5 to 6 carbon atoms (C₅-C₆ cycloalkyl).

Alkyl or cycloalkyl groups as used herein may optionally include further substituent groups. A substitution on the cycloalkyl group also encompasses an aryl, a heterocycle or a heteroaryl substituent, which can be connected to the cycloalkyl group via one atom or two atoms of the cycloalkyl group.

As used herein the term “alkenyl,” refers to a straight or branched hydrocarbon chain moiety containing in particular up to 6 carbon atoms and having at least one carbon-carbon double bond. Examples of alkenyl groups include, without limitation, ethenyl, propenyl, butenyl, 1-methyl-2-buten-1-yl, dienes such as 1,3-butadiene and the like. Alkenyl groups as used herein may optionally include further substituent groups.

As used herein the term “alkynyl,” refers to a straight or branched hydrocarbon moiety containing in particular up to 6 carbon atoms and having at least one carbon-carbon triple bond. Examples of alkynyl groups include, without limitation, ethynyl, 1-propynyl, 1-butynyl, and the like. Alkynyl groups as used herein may optionally include further substituent groups.

As used herein the term “heterocycle” refers to an interconnected alkyl group forming a saturated or unsaturated ring or polyring structure containing 3 to 10, particularly 5 to 6 carbon atoms in which at least one carbon atom is replaced with an oxygen, a nitrogen or a sulphur atom forming a nonaromatic structure. Due to simplicity reasons they are denominated e.g. C₅ to C₁₀ heterocycle, wherein at least one carbon atom is replaced with an oxygen, a nitrogen or a sulphur atom forming a ring structure. Heterocyclic groups as used herein may optionally include further substituent groups. A substitution on the heterocyclic group also encompasses an aryl, a cycloalkyl or a heteroaryl substituent, which can be connected to the heterocyclic group via one atom or two atoms of the heterocyclic group (comparable to indole).

As used herein the term “aryl” refers to a hydrocarbon with alternating double and single bonds between the carbon atoms forming an aromatic ring structure, in particular a six (C₆ to ten (C₁₀) membered ring or polyring structure, in particular a six membered ring.

The term “heteroaryl” refers to aromatic structures comprising a five to ten membered ring or polyring structure, in particular five to six membered ring structure, comparable to aryl compounds, in which at least one member is an oxygen or a nitrogen or a sulphur atom. Due to simplicity reasons they are denominated e.g. C₅ to C₁₀ heteroaryl, wherein at least one carbon atom is replaced with an oxygen, a nitrogen or a sulphur atom forming an aromatic structure. For example a C₅ heteroaryl comprises a five membered ring structure with at least one carbon atom being replaced with an oxygen, a nitrogen or a sulphur atom.

Aryl or hetero aryl groups as used herein may optionally include further substituent groups. A substitution on the hetero aryl group also encompasses an aryl, a cycloalkyl or a heterocycle substituent, which can be connected to the hetero aryl via one atom or two atoms of the hetero aryl group (comparable to indole). The same applies to an aryl group.

### BRIEF DESCRIPTION OF THE FIGURES

FIG. 1 shows optimization of the phenylimino-thiazolidinone hit 7 to the selective Aurora A inhibitor 9. (a) Structure and activities of analogs with variations of R¹ and R². (b) Optimization of the 2-pyridyl substituent. SI=selectivity index: IC₅₀(Aurora B/INCENP)/IC₅₀(Aurora A).

FIG. 2 shows crystal structures of the 4-thiazolidinone derivatives bound to Aurora A. The 2Fₒ-F_{c} electron density map contoured at 1 sigma around the compounds is shown to highlight their unambiguous position in the pocket. The crystal structures refer to PDB codes 4ZTQ, 4ZTR and 4ZTS, resolution 2.8-2.9 Å. The catalytic pocket of Aurora A acquires an inactive conformation upon binding to 77 (a), 9 (b) and 88 (c). The lower panels show schematic representations of the interactions between the compounds and the protein. Dashed lines indicate hydrogen bonds, in particular interactions between the aminopyridine portion of the inhibitors and the hinge region (A213) and between the anionic carboxyl or tetrazole group and the guanidinium groups of R137 and R220. Residues involved in hydrophobic interactions are highlighted with an arc.

FIG. 3 shows that 9 impairs localization of Aurora A at the mitotic spindle. Representative images of HeLa Kyoto cells treated with compound 9 overnight and stained for DNA (blue), Aurora A (green) and TPX2 (red). TPX2 co-localizes with Aurora A in control cells (top row). 9 impairs Aurora A localization at the spindle microtubules but does not affect centrosomal Aurora A. Aurora A is also present at centrosomes but not on spindle microtubules in cells treated with 1 (MLN8237).

FIG. 4 shows that inhibitor 9 yields an Aurora A specific inhibition phenotype in cells without impairing Aurora B function. (a) Representative images of HeLa Kyoto cells after overnight incubation with indicated compounds. The cells were stained for pThr288 Aurora A (red), α-tubulin (green), and DNA (blue). Impaired alignment of chromosomes on the metaphase plate is indicated by white arrows. (b) Representative images of HeLa Kyoto cells after overnight incubation with indicated compounds and staining for pHis-H3 (red) and DNA (blue). (c) Western blot of lysates from cells synchronized in prometaphase with 100 nM nocodazole and treated with the indicated inhibitors. β-actin was used as loading control. (d) DNA content analysis of cells treated for 48 h with the indicated compounds. The cells were stained with propidium iodide and analyzed using flow cytometry.

FIG. 5 shows in vitro reactivity of 9 towards glutathione (GSH). Inhibitor 9 (30 μM) was incubated with GSH (5 mM) in H₂O/CH₃CN (4:1) at pH=7.0, and the reaction was followed by LC-MS. a. Reaction scheme and structures of modified analogs. b. LC chromatogram of the reaction mixture after t=0 h and t=14 h. The product formed with a retention time tᵣₑₜ=1.29 min is identified by MS as the GSH-adduct of 9. 9-methylcarbazole (tᵣₑₜ=2.44 min) was used as internal control. c. Concentration of compound 9 during the course of the reaction as determined by integration of the absorption peaks (at 310 nm) of compound 9 at tᵣₑₜ=1.78 min. The average of two independent experiments is shown, SD is <5% for all data points. d. Aurora A inhibition in the HTRF biochemical assay. (d) Cellular pThr288 Aurora-A levels of cells incubated with 9 or rac-92 overnight at the indicated concentrations. The experiment was repeated twice and the error bars represent SD. Derivative rac-92 does not reduce pThr288 levels in cells, although having an IC₅₀ of 24 nM in the biochemical assay. Numbers represent average IC₅₀±SD of two independent experiments.

FIG. 6 shows the structures and IC₅₀ values of known Aurora inhibitors.

### EXPERIMENTAL SECTION

The compounds disclosed herein are synthesized according to Kilchmann et al. (Kilchmann et al., J Med Chem 59, 7188-7211, in particular reference is made to schemes 1 to 4, to the paragraph “Synthesis” on page 7191 and to the experimental section on page 7197pp.) The reaction schemes (2) and (3) are a summary of the reactions described in Kilchmann et al. (Kilchmann et al., J Med Chem 59, 7188-7211). Comparable compounds can be synthesised analogously.

The compounds of formula (1a) are synthesized according to reaction scheme (2). The temperatures and reaction times are optimal for compounds wherein R¹ is methyl and R² is ethyl. For other substituents, it might be necessary to adjust the temperatures and reaction times appropriately or change other parameters accordingly.

The reaction steps of scheme (2) are as follows:

Step 1: A solution of R¹ₙ-aniline in EtOH is treated with R²-isothiocyanate and stirred at 78° C. for 12 h. The solution is then treated with NaOAc and ethyl chloroacetate and stirred at 78° C. for another 5 h. EtOH is evaporated in vacuo to afford the crude product as oil.
Step 2: A solution of the crude reaction product of step 1 and piperidine in EtOH is treated with tert-butyl(4-formyl-pyridine-2-yl)carbamate and stirred at 75° C. for 15 h. The solution is cooled to 0° C. and filtered. The solid on the filter is washed with ice-cold EtOH and added to TFA. After 4 h, the solution is diluted with H₂O and CH₂Cl₂ and neutralized with NaOH. The organic phase is washed with H₂O, dried over MgSO₄-2H₂O and evaporated in vacuo to afford a solid product.
Step 3a: To obtain a compound wherein L is an alkyl, a solution of the reaction product of step 2 and pyridine is treated with Boc-protected aminocarboxylic acid (e.g. N-(tert-butoxycarbonyl)-3-aminopropionic acid) and stirred overnight. Then, the solution is treated with EtOAc, washed with H₂O and evaporated in vacuo. The crude product is redissolved in TFA/THF (1:1) and stirred for 5 h. The mixture is then diluted with H₂O and neutralized with NaOH. The organic phase is washed with H₂O, dried over MgSO₄.2H₂O and evaporated in vacuo to afford a solid product after lyophilization. To obtain carboxylic acids, a tert-butyl-carboxylic acid might be used (e.g. tert-butyl malonate).

Alternatively, a solution of the reaction product of step 2, pyridine and the corresponding anhydride to R⁴ is used. For example, succinic anhydride is added and stirred for 12 h. After adding EtOAc, the organic phase is washed with 1 M HCl, brine and H₂O. The solvent is evaporated in vacuo and the crude product purified by RP-HPLC to afford a solid product.

In case of glutaric anhydride the solution of the reaction product of step 2, pyridine and the anhydride is heated to 100° C. for 1 h. The precipitate is filtered and washed with EtOAc and H₂O. The product is dried on high vacuum overnight to afford a solid product.

Other compounds can be produced analogously.

Step 3b: To obtain a compound wherein L is an aryl or heteroaryl (both are referred to in the scheme as “Ar”) degassed dioxane is added to a flask containing the product of step 2, Pd₂(dba)₃, Xantphos, Cs₂CO₃, and Ar—Br under argon. The suspension was degassed and refilled with argon (5×) and then heated to 100° C. The reaction mixture is stirred overnight at 100° C., then cooled to room temperature, filtered, and concentrated in vacuo. The crude is purified by FC to afford a solid product. Other compounds can be produced analogously.

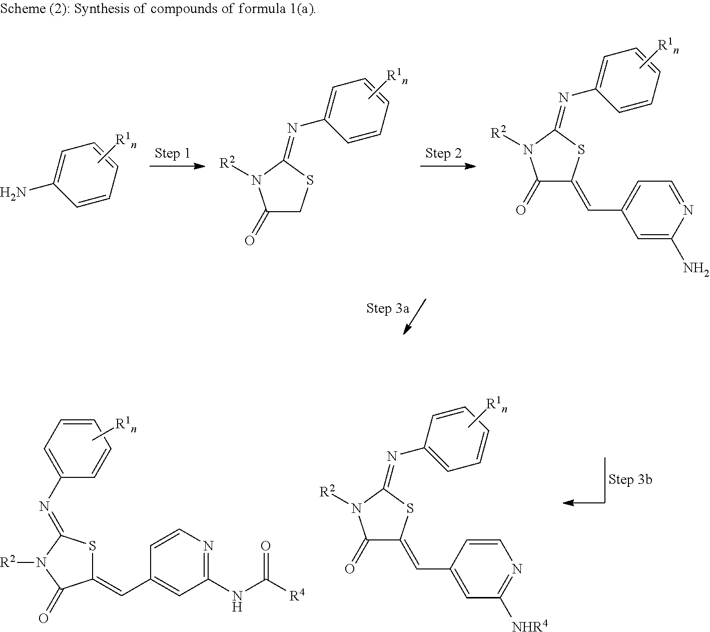

Compounds of formula (1b) are synthesized according to reaction scheme (3). The reaction starts with a molecule synthesized according to scheme (2).

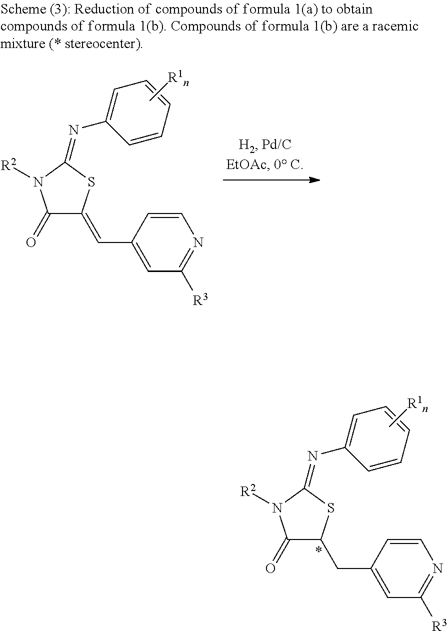

### EXAMPLES

### Thiazolidinone Inhibitors

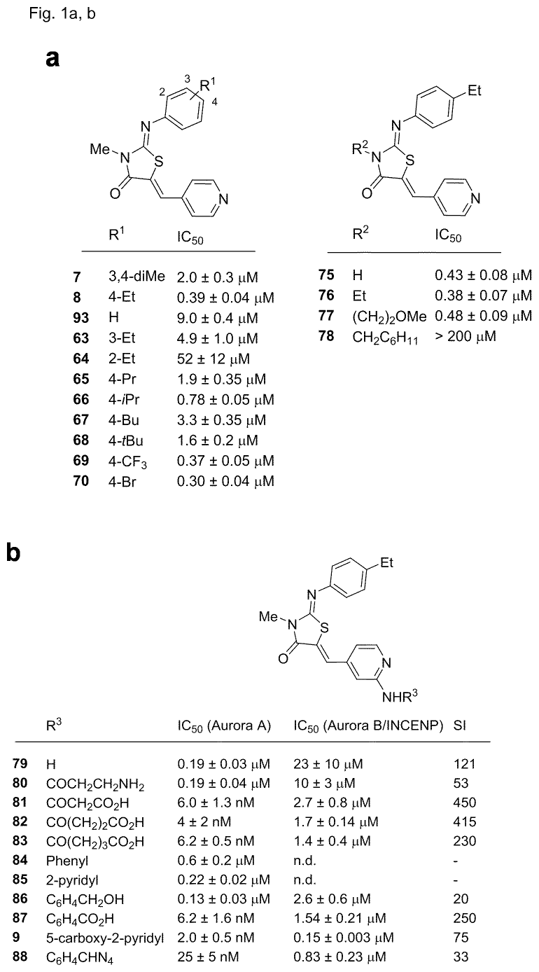

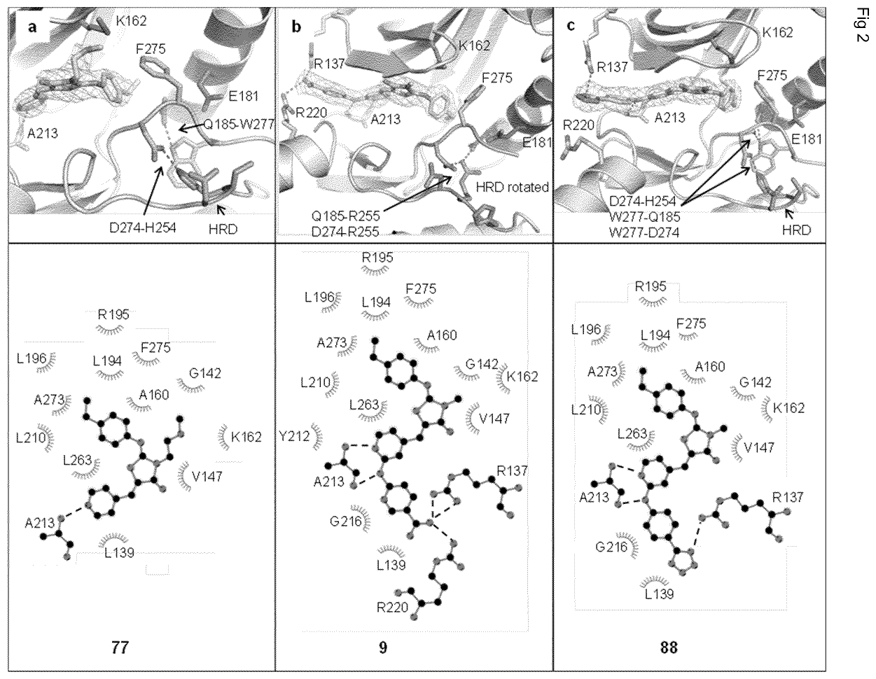

The inventors investigated phenylimino-thiazolidinones, starting with compound 7 (FIG. 1a). Initial SAR profiling indicated potential for optimization upon variation of the N-phenyl substituents with 8 (FIG. 1a) and pinpointed to the essential role of the 4-pyridine ring since several analogs of 7 with alkyl, halogen, hydroxy or methoxy substituted phenyl rings or a 3-pyridyl ring at that position were inactive. Additional analogs were investigated to test if the initial gain in activity with 8 could be improved further (FIG. 1a). Activity profiling confirmed the optimal 4-ethylphenylimino substituent of 8 (63-70 and 93, FIG. 1a) and the need for a small alkyl substituent on the endocyclic nitrogen atom of the thiazolidinone (75-78, FIG. 1a). Furthermore, crystal structures of 76 and 78 established the Z-stereochemistry of both exocyclic double bonds of the thiazolidinone core.

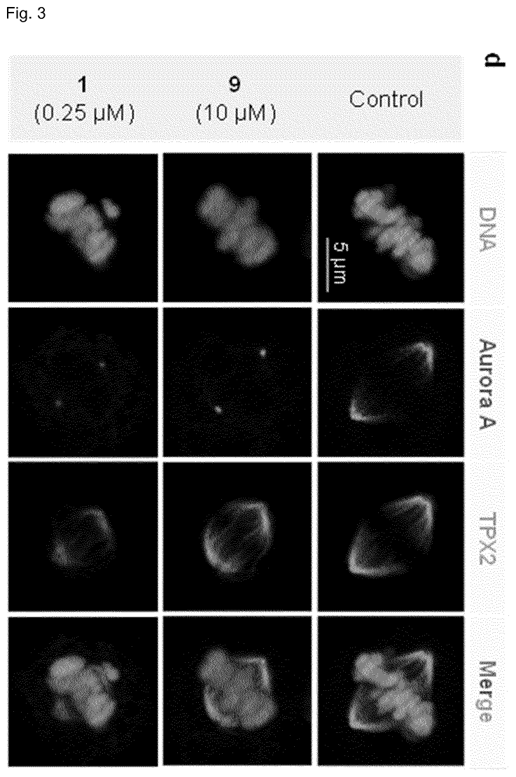

A further crystal structure of 77 in complex with Aurora A (FIG. 2) suggested that placing an amino group at the 2-pyridyl position substituted with acyl or aromatic groups might enhance hinge-binding interactions. Significant potency gains were indeed obtained by this approach, reaching low nanomolar values with 81-83 and 87, 9 and 88 displaying substituents with a carboxylate or tetrazole group presumably interacting with Arg137 (FIG. 1b).

These inhibitors were remarkably selective for Aurora A against Aurora B used in complex with its physiological activator INCENP. The most potent inhibitor 9 (IC₅₀=2±0.5 nM for binding to Aurora A, IC₅₀=149±3 nM for binding to Aurora B in complex with INCENP) was selected for further in depth analysis because this compound was also the most potent when tested on cells (see below). A kinome scan of 456 kinases using an active site directed binding competition assay (Fabian et al., Nat Biotechnol, 2005, 23, 329-336) followed by determination of binding affinities showed that 9 was remarkably selective and bound tightly only to Aurora A (K_{D}=5 nM), Aurora B (without INCENP, K_{D}=10 nM), and Aurora C (K_{D}=11 nM) (Karaman et al., Nat Biotechnol, 2008, 26, 127-132). Note that the biochemical inhibition and binding affinity measurements were both carried out with free Aurora A, which is autophosphorylated at T288, and gave comparable values. By contrast the biochemical inhibition of Aurora B was measured for its complex with its activator protein INCENP due to the inactivity of the kinase alone, giving a 15-fold weaker inhibition compared to its K_{D} value measured with free Aurora B.

### Binding of 9 to Aurora A Excludes the Activator Protein TPX2

To understand the inhibition mechanism at the atomic level, the inventors solved the crystal structures of Aurora A in complex with 77 from the initial SAR study, with the highest affinity inhibitor 9, and with its phenyl tetrazole analog 88 (PDB codes 4ZTQ, 4ZTR and 4ZTS, resolution 2.8-2.9 Å, FIG. 2). All three inhibitors bind to the ATP pocket in the adenine binding region (Leu139, Val1147, Ala160, Leu263) and occupy the hydrophobic back pocket (Leu194, Arg195, Leu196, Leu210, Phe275). The inhibitors induce an inactive DFG motif conformation also found in other Aurora A inhibitor complexes (PDB codes 4JBQ, 4JAI, 2J5O, 2BMC, 3P9J, 3R22, 3FDN, 3K5U, and 4BOG) with four characteristic features: (1) disruption of the R-spine (residues Leu196, Gln185, Phe275, His254, and Asp311), (2) an outward displacement of the aC helix compared to the active state (approximately 2.5 Å), (3) absence of the salt bridge between Lys162 and Glu181, and (4) disruption of the Asp256-Thr292-Lys258 hydrogen bond network.

In this conformation Asp274 is pointing away from the ATP pocket and Phe275 is rotated upwards thereby contacting the phenylimino moiety of the inhibitors. Furthermore, Trp277 (or Arg255 of the HRD motif in the case of 9) forms H-bonds with Gln185 and Asp274, an arrangement clearly different from classical DFG-in or DFG-out conformations, and which is specific of Aurora kinases, thus probably contributing to inhibitor selectivity. The pyridine/aminopyridine group of the inhibitors engages in one or two hydrogen bonds with Ala213 in the hinge region. Finally, the terminal carboxylate of 9 forms a salt bridge with Arg137 and Arg220, analogous to alisertib in the structure bound to Aurora A (PDB code 2X81), thus explaining its stronger affinity compared to 77.

In Aurora A, the DFG motif is followed byTrp277. Tryptophan fluorescence experiments showed that binding of 9 and of its analogs 77 and 88 perturbed the environment of Trp277, reflecting the rearrangement of the DFG loop and Trp277 interactions with Gln185 occurring upon binding of these inhibitors, as suggested by inspection of the crystal structures. This indicates that the DFG motif rearrangement also occurs in solution. Further analysis of these crystal structures indicated that this rearrangement should be incompatible with the rotation of the aC-helix and the subsequent salt bridge formation between Lys162 and Glu181 that take place upon binding to the microtubule-associated activator protein TPX2 (PDB code 1OL5 or 4C3P).

To test this hypothesis, the inventors measured the binding of Aurora to labeled TPX2¹⁻⁴³ using microscale thermophoresis (K_{D}=795±129 nM). The inventors found in three independent measurements that this binding was indeed abolished in the presence of excess 9. The incompatibility between 9 and TPX2 was further evidenced by the fact that TPX2 binding, which induced a 3-fold increase in the activity of Aurora A similar to other reports, reduced inhibition by 9 from IC₅₀=2.0 nM to IC₅₀=1.4 μM. In control human cells, TPX2 helps in the localization of Aurora A to the spindle microtubules, but not to the centrosomes. These findings raised the possibility that addition of 9 should prevent localization from taking place strictly on the spindle microtubules. Indeed, the inventors found that Aurora A remained localized at centrosomes in the presence of 9 but was notably displaced from the spindle microtubules, as is the case for alisertib (1) (FIG. 3).

### Compound 9 Selectively Inhibits Aurora A in Cells

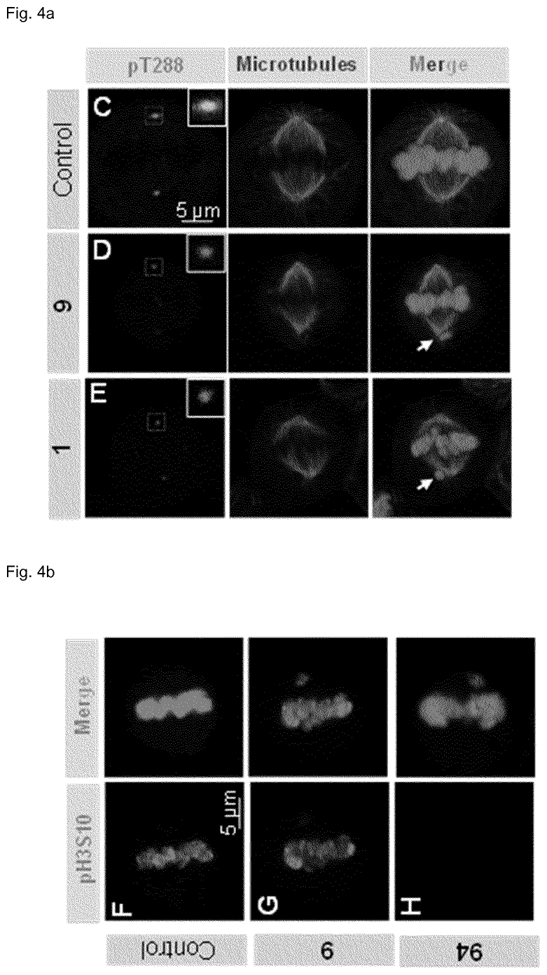

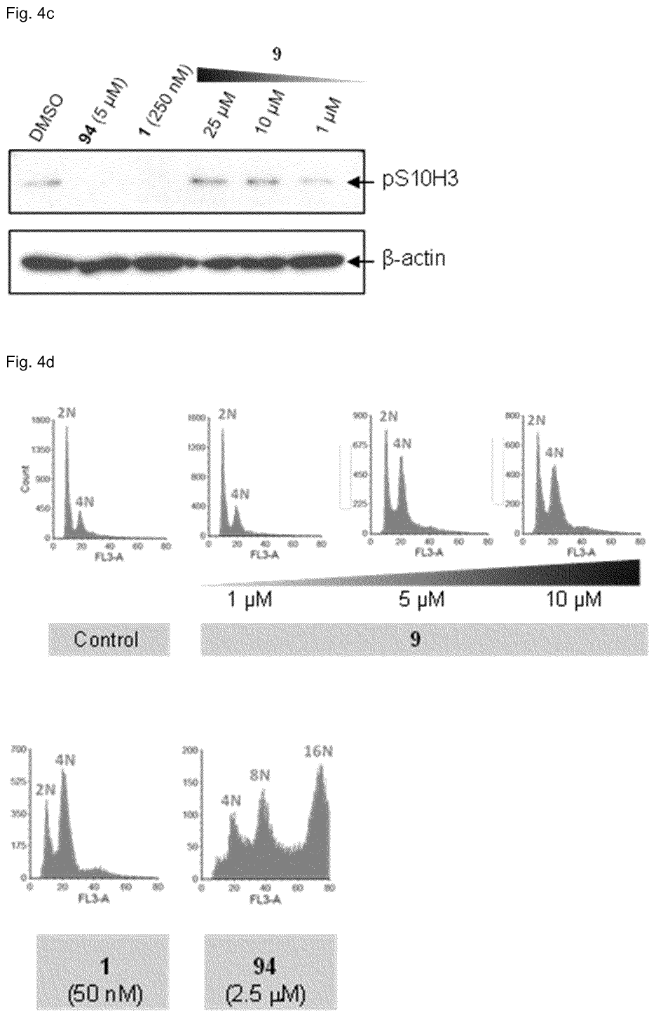

The inventors set out to test the impact of 9 on mitotic progression of human tissue culture cells. Treatment of HeLa cells with 9 resulted in a dose-dependent increase in the mitotic index, as observed also with the reference inhibitor 1 (alisertib) and as expected from the role of Aurora A for timely progression through mitosis. The mitotic index increased from ˜15% at 4 μM 9, ˜25% at 10 μM 9 to ˜40% at 25 μM 9. 0.25 μM alisertib resulted in a mitotic index of ˜30%. Inhibitor 9 also decreased phosphorylation of Aurora A Thr288, an autophosphorylation site that marks Aurora A kinase activity (IC₅₀=760 nM). Furthermore, 9 induced defective chromosome alignment during metaphase, with a phenotypic EC₅₀ value of 6 μM, in line with the requirement of Aurora A activity for proper spindle dynamics (FIG. 4a). ˜20% misaligned chromosomes in HeLa Kyoto cells were observed at 4 μM inhibitor 9. ˜80% and ˜85% misaligned chromosomes were detected at 10 μM and 25 μM 9, respectively. Almost 100% misaligned chromosomes were observed at 0.25 μM alisertib.

Many Aurora A kinase inhibitors also target Aurora B in the cellular context. To address whether this may be the case also for 9, the distribution of phospho-Histone H3 Ser10, a histone modification that is imparted during early mitosis by Aurora B, was examined. The inventors found that this feature remained unchanged in cells treated with 9, whereas it was absent in cells treated with the Aurora B inhibitor ZM447439 (94) (FIG. 4b/c) (Ditchfield et al., J Cell Biol, 2003, 161, 267-280). Furthermore, the massive accumulation of cells with 8N and 16N DNA contents observed by flow cytometry upon treatment with 94, which is a hallmark of defective cytokinesis following Aurora B inactivation, also did not occur with 9 even when provided at 10 μM (FIG. 4d). The inventors conclude that in contrast to other Aurora A inhibitors, including the reference inhibitor alisertib, the inhibitor 9 had no effect on Aurora B activity in cells.

To unequivocally test whether the effect of 9 on human cells was specific to Aurora A inhibition, the inventors performed phenotypic rescue experiments using cells expressing GFP-Aurora A-Arg137Ala or GFP-Aurora A-Trp277Ala mutants, which were predicted by examination of the crystal structure to be insensitive to 9. The inventors found that these proteins were insensitive to the addition of 9, demonstrating that the phenotype normally induced by this compound was indeed caused by Aurora A inhibition.

Compound 9 Shows Moderate Reactivity with Glutathione

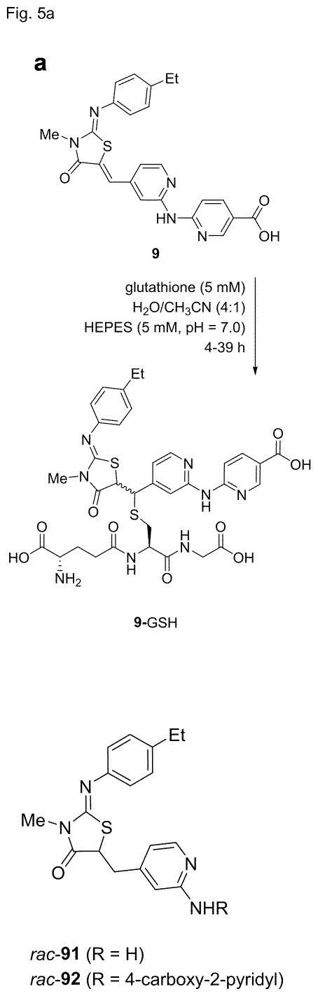

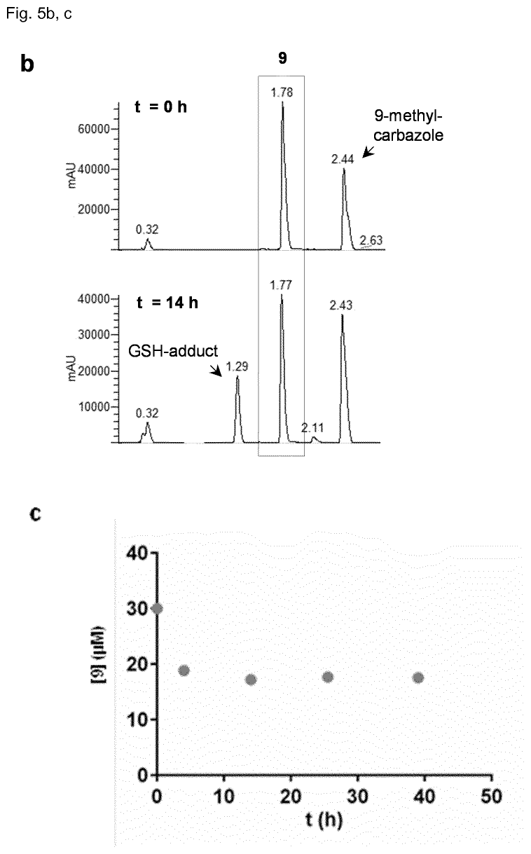

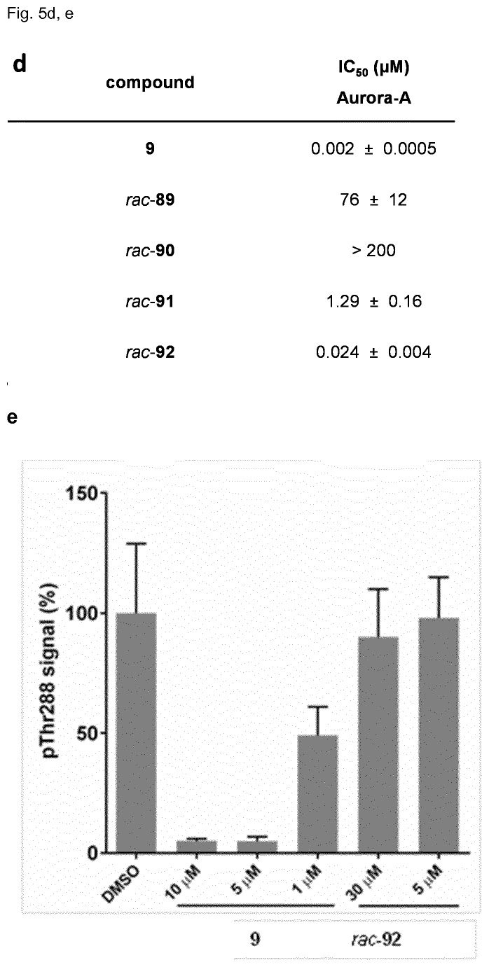

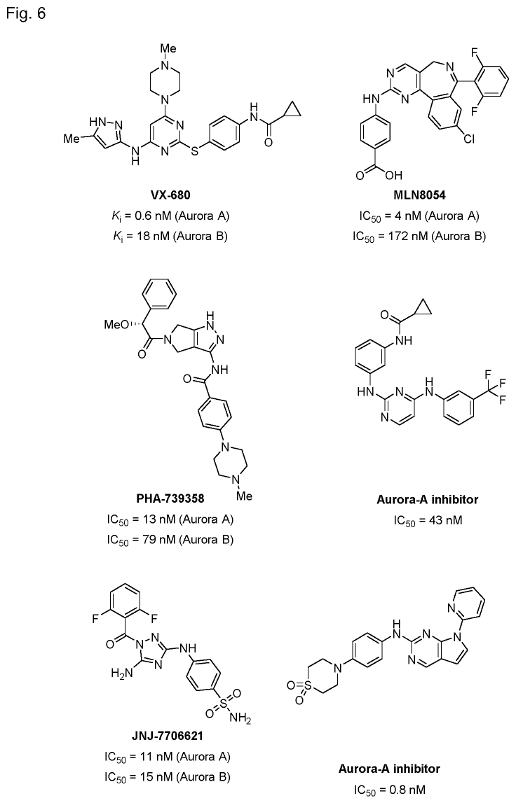

Despite of its high selectivity towards Aurora A, the cellular activity of 9 (EC₅₀>1 μM) was much weaker than its biochemical activity (IC₅₀=2 nM). To understand whether this lower activity might be caused by covalent reaction of the electrophilic double bond of 9, the inventors measured its reactivity towards the intracellular nucleophile glutathione (GSH) under physiological conditions. Conversion to a GSH adduct was indeed detected, however only to a limited extent (50% conversion after 24 h with 5 mM GSH, pH 7.4, 37° C., FIG. 5), suggesting that a significant portion of the inhibitor remained unreacted within the cell and was available for inhibition of Aurora A. Nevertheless analogs of 9 lacking the electrophilic double bond were investigated as alternatives. Diastereomeric cyclopropanes rac-89/rac-90 were more than 400-fold less active that their precursor 79. On the other hand its reduced double bond analog rac-91 was only 7-fold less active, and the reduced double bond analog of 9, rac-92, was also still quite potent (IC₅₀=24 nM). However this derivative did not show any activity in cells despite the fact that it cannot form GSH adducts.

Taken together, these data suggest that GSH reactivity is insufficient to explain the discrepancy between the IC₅₀ values of cellular versus biochemical activity of 9 on Aurora A and B presented above (Aurora A: biochemical IC₅₀=2.0 nM, cellular IC₅₀ (pT288)=760 nM, Aurora B/INCENP: biochemical IC₅₀=149 nM, cellular IC₅₀ (pH3)>25 μM). This activity difference is probably a consequence of the higher ATP concentration in cells (1 mM) compared to in vitro assay (20 μM) and the presence of competing ligands such as TPX2 (FIG. 4d), as well as the presence of a carboxylate group which might reduce cellular uptake. The same effects probably explain the weaker cellular versus biochemical activities reported for inhibitor 1 (Aurora A: biochemical IC₅₀=0.04 nM, cellular IC₅₀ (pT288)=6.7 nM, Aurora B/INCENP: biochemical IC₅₀=1.1 nM, cellular IC₅₀ (pH3)=1.5 μM), as well as for MK-5108, a further selective Aurora A inhibitor (Aurora A: biochemical IC₅₀=0.064 nM, cellular IC₅₀ (pT288)=300 nM, Aurora B/INCENP: biochemical IC₅₀=1.49 nM, cellular IC₅₀ (pH3)>10 μM).

### Materials and Methods

### Chemistry.

All reagents were purchased from commercial sources and were used without further purification. Flash chromatography purifications were performed with silica Gel 60 (Fluka, 0.040-0.063 nm, 230-400 mesh ASTM). Low resolution mass spectra were obtained by electron spray ionization (ESI-MS) in the positive mode on a Thermo Scientific LCQ Fleet. High resolution mass spectra were obtained by electron spray ionization (HR-ESI-MS) in the positive mode recorded on a Thermo Scientific LTQ Orbitrap XL. ¹H and ¹³C-NMR spectra were measured on a Bruker Avance 300 spectrometer (at 300 MHz and 75 MHz, respectively) or on a Bruker AVANCE II 400 spectrometer (at 400 MHz and 101 MHz, respectively). ¹H and ¹³C chemical shifts are quoted relative to solvent signals, and resonance multiplicities are reported as s (singlet), d (doublet), t (triplet), q (quartet), p (pentet), and m (multiplet); br=broad peak. Compound purities were assessed by analytical reversed phase HPLC (RP-HPLC) at a detection wavelength of 254 nm or 310 nm. The purity of tested compounds was >95% for all compounds. Analytical RP-HPLC was performed on a Dionex Ultimate 3000 RSLC System (DAD-3000 RS Photodiode Array Detector) using a Dionex Acclaim RSLC 120 column (C18, 3.0×50 mm, particle size 2.2 μm, 120 Å pore size) and a flow rate of 1.2 mL min⁻¹. Data were recorded and processed with Dionex Chromeleon Management System (version 6.8) and Xcalibur (version 2.2, Thermo Scientific). Eluents for analytical RP-HPLC were as follows: (A) milliQ-deionized water containing 0.05% TFA, and (D) HPLC-grade acetonitrile/milliQ-deionized water (9:1) containing 0.05% TFA. Conditions for analytical RP-HPLC were as follows: From A/D (7:3) to 100% D (2.2 min) followed by 100% D (1 min). Preparative RP-HPLC was performed with a Waters Prep LC4000 Chromatography System using a Reprospher 100 column (Dr. Maisch GmbH, C18-DE, 100×30 mm, particle size 5 μM, pore size 100 Å) and a Waters 489 Tunable Absorbance Detector operating at 214 nm. Eluents for preparative RP-HPLC were as follow: (A) milliQ-deionized water containing 0.1% TFA, and (D) HPLC-grade acetonitrile/milliQ-deionized water (9:1) containing 0.1% TFA.

Compounds 1, 7, 9, 63-70, 75-81, 84-86, 88, 93 and rac-89-92 were analysed as follows: From A/D (7:3) to 100% D (2.2 min) followed by 100% D (1 min); detection at 254 nm. The retention times of these compounds ranged from 1.02 min to 2.33 min.

The compounds 8, 82, 83 and 87 were analysed as follows: From A/D (7:3) to 100% D (2.2 min) followed by 100% D (1 min); detection at 310 nm. The retention times of these compounds ranged from 1.43 min to 1.85 min.

The compounds depicted in FIG. 1 were synthesized according to Kilchmann et al. (Kilchmann et al., J Med Chem 59, 7188-7211, in particular reference is made to schemes 1 to 4, to the paragraph “Synthesis” on page 7191 and to the experimental section on page 7197pp.)

### Aurora A Purification.

The clone of human Aurora A kinase domain (residues 122-403 in the pET24-d vector) was kindly provided by the Montoya laboratory (University of Copenhagen). The construct was transformed into E. coli BL21 (DE3) Rosetta cells and protein expression was induced with 0.5 mM IPTG at 20° C. for 12 hours. The cells were harvested at 6,000×g for 25 min at 4° C. and resuspended in lysis buffer (50 mM Tris-HCl, 500 mM NaCl, 1 mM PMSF, pH=8.0). Disruption of the cells was performed by sonication cooled on ice, after which the debris were removed by centrifugation at 110,000×g for 30 min at 4° C. Aurora A was purified by affinity chromatography using NiNTA resin from Qiagen following the manufacturer's instructions. After loading, the resin was washed with lysis buffer, followed by a second wash with 6% elution buffer (50 mM Tris-HCl, 500 mM NaCl, 500 mM imidazole, pH=8.0). Protein was eluted with 100% elution buffer. The eluate was exchanged into final buffer (20 mM Tris-HCl, 200 mM NaCl, 0.5 mM EDTA, 2 mM DTT, pH=8.0) using a HiPrep 26/10 desalting column (GE Healthcare). The His-tag was cleaved with TEV protease at 4° C. overnight. The tag and minor impurities were removed by a second nickel affinity chromatography step. Aggregated and soluble Aurora A were separated from one another by size exclusion chromatography using a HiLoad 16/60 Superdex™ 200 column (GE Healthcare) equilibrated in final buffer. Soluble Aurora A was concentrated using Vivaspin-15 concentrators (Sartorius Stedim Biotech). Protein concentration was determined by UV absorbance. Aurora A was flash frozen in liquid nitrogen and stored at −18° C.

### HTRF Kinase Assay.

Aurora A and AuroraB kinases were assayed using the homogeneous time-resolved fluorescence (HTRF) KinEASE STK2 kit from Cisbio (France). For Aurora A, which was expressed and purified as described above, the enzymatic reaction (total volume 10 μL) was carried out with 3 nM Aurora A kinase domain, 1 μM biotinylated STK2 substrate, 20 μM ATP (˜Kₘ) in kinase buffer (50 mM HEPES (pH 7.0), 0.02% NaN₃, 0.1 mM Na₃VO₄, 0.01% BSA, 5 mM MgCl₂, 0.01% Triton X-100, 1 mM DTT), and either the test compound or the DMSO control (final DMSO concentration was 2%). For AuroraB, the enzyme reaction (total volume 10 μL) was carried out with 8 nM AuroraB/INCENP complex (Millipore, no. 14-835), 1 μM biotinylated STK2 substrate, 20 μM ATP (Kₘ=26 μM) in kinase buffer (50 mM HEPES (pH 7.0), 0.02% NaN₃, 0.1 mM Na₃VO₄, 0.01% BSA, 5 mM MgCl₂, 0.01% Triton X-100, 1 mM DTT), and either the test compound or the DMSO control (final DMSO concentration was 2%). For both enzymes, the reactions were run for 30 min at room temperature and stopped by the addition of 10 μL of detection buffer containing EDTA, antiphospho-Ser/Thr antibody labelled with europium cryptate, and XL-665 conjugated streptavidin (62.5 nM final concentration). After incubation at room temperature for one hour, fluorescence was measured at 620 nm (europium cryptate) and 665 nm (XL-665) after excitation at 317 nm (lag time 60 μs, integration time 500 μs) using a Tecan Infinite M1000 PRO microplate reader (Greiner 384-well plates, white, non-binding, small volume). The ratio of fluorescence (665 nm/620 nm) was calculated for each well and the results were expressed as follows: specific signal=ratio (sample)−ratio (negative control), where ratio=665 nm/620 nm×10⁴. Compounds were measured in threefold serial dilutions at 10 different concentrations, covering the concentration range from no to full inhibition. Each concentration was measured in duplicates, and two independent determinations were made for each IC₅₀ value. IC₅₀ curves were generated using a four-parameter logistic model (XLfit from IDBS).

### Kinome Profiling.

The kinome profiling of compound 9 was conducted by DiscoveRx. In total, 456 kinases were assayed (scanMAX). For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6000 g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.04 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR. Compound 114 was screened at 1 μM, and results for primary screen binding interactions were reported as % of control (PoC). PoC=(test compound signal−positive control signal)/(DMSO signal−positive control signal). PoC values at 1 μM 9 for all kinases were visualized using TREEspot (DiscoveRx). For the determination K_{D} values, an 11-point 3-fold serial dilution of 9 was prepared in 100% DMSO at 100× final test concentration and subsequently diluted to 1× in the assay (final DMSO concentration=1%). K_{D} values were determined with a standard dose-response curve using the Hill equation.

### Crystallization, Structure Solution and Refinement.

Aurora A kinase domain was concentrated to 150 μM and mixed with MgSO₄ and the corresponding compound (dissolved in DMSO) to a final concentration of 2 mM and 150 μM, respectively. Crystallization experiments were set up after incubation of the complex for 15 min on ice. The crystals were obtained by hanging drop vapour diffusion at 18° C., mixing 1 μl of sample solution with 1 μl of reservoir solution and equilibrating against 500 μl of reservoir solution. The crystals were flash frozen in liquid nitrogen under cryo-protection. The crystallization solutions found for each complex were: 0.3 M Ammonium citrate dibasic, 25% PEG 3350 (10% MPD added as cryo-protectant) for 77/Aurora A; 0.1 M Tris pH 8.0, 28% PEG 4K (20% glycerol added as cryo-protectant) for 9/Aurora A; and 5% MPD, 0.1 M Hepes pH 7.5, 10% PEG 10K (20% MPD added as cryo-protectant) for 88/Aurora A.

Data were collected on beamline X06DA at the Swiss Light Source, Paul Scherrer Institut, Villigen, Switzerland, except the data set for the 77/Aurora A complex that was collected on beamline X06SA, also at the Swiss Light Source. The wavelength was 1 Å and the temperature 100 K. The data were integrated and scaled with MOSFLM/SCALA (Evans, Acta Cryst D, 2006, 62, 72-82; Leslie et al., Nato Sci Ser II Math, 2007, 245) or XDS (Kabsch, W Xds Acta Cryst D, 2010, 66, 125-132). The structures were solved by molecular replacement using Phaser in CCP4 (Winn et al., Acta Cryst D, 2011, 67, 235-242) and PDB 1OL5 without ligand as template. All complexes crystallized in the P6₁22 space group with one molecule per asymmetric unit. A first round of rigid body refinement was performed, followed by simulated annealing (Adams et al., Acta Cryst D, 2010, 66, 213-221) and initial electron density maps showed the presence of the compounds. These were fitted using LigandFit in Phenix (Adams et al., Acta Cryst D, 2010, 66, 213-221). Several rounds of refinement (Phenix and Refmac5 (Winn et al., Acta Cryst D, 2011, 67, 235-242) (using TLS) and model building (Coot)(Emsley et al., Acta Cryst D, 2010, 66, 486-501) were subsequently carried out until convergence was reached. The final models have no Ramachandran outliers and they lack 8 and 15 residues at the N- and C-terminus, respectively. In addition, in the complex structures of Aurora A with 77, 9 and, 88 structures, 13 residues in the activation loop were not visible (residues 279-291). The figures were compiled using Pymol (DeLano, The PyMOL Molecular Graphics System (2002), on World Wide Web http://www.pymol.org) and Ligplot (Wallace et al., Protein Eng, 1995, 8, 127-134).

### TPX2 Purification.

The clone of human TPX2 N-terminal domain (residues 1-43), preceded by GST and a TEV cleavage site, was a generous gift from the Conti laboratory (MPI, Martinsried). The protein was expressed in E. coli Bl21 (DE3) cells using 0.1 mM IPTG at 20° C. for 12 hours. Cells were broken and the soluble fraction was isolated as explained above. TPX2 was purified using a GSTrap (GE Healthcare) equilibrated in 50 mM Tris, 150 mM NaCl, pH=7.5. GST-TPX2 was eluted using final buffer plus 10 mM reduced glutathione. The GST-tag was cleaved with TEV protease at 4° C. overnight. TPX2 (1-43) was isolated in a final step of size exclusion chromatography using a HiLoad 16/60 Superdex™ 75 column (GE Healthcare) equilibrated in final buffer. Protein concentration was determined by UV absorbance. TPX2 was flash frozen in liquid nitrogen and stored at −18° C.

### Microscale Thermophoresis.

TPX2 was labelled using the Monolith NT.115 protein labelling kit RED (NanoTemper Technologies). The labelling was performed according to the manufacturer's instructions in the supplied labelling buffer applying a concentration of 44 μM peptide at room temperature for 30 min. Labelled TPX2 was adjusted to 400 nM with final buffer and 0.05% Tween-20 (NanoTemper Technologies). Aurora A kinase with or without ligand was dissolved in final buffer supplemented with 0.05% Tween-20, and a series of 12 dilutions (1:1) was prepared in the identical buffer, keeping the DMSO/ligand concentration constant (final concentrations 0.5% and 125 μM, respectively). Final protein concentrations were between 4.4 nM and 150 μM. For the thermophoresis experiment, each protein dilution was mixed with one volume of labelled TPX2, which led to a final concentration of 200 nM for fluorescently labelled TPX2, 125 μM for the ligand and 2.2 nM to 75 μM for Aurora A. After 30 min incubation at 4° C. and centrifugation at 9,600×g for 2 min, the solution was filled into Monolith NT hydrophilic capillaries (NanoTemper Technologies). Thermophoresis was measured at room temperature with 5 s/30 s/5 s laser off/on/off times. Instrument parameters were adjusted to 15% LED intensity and 80% MST power. Data of three independent measurements were analyzed (NT.Analysis software, version 1.5.41, NanoTemper Technologies) using the thermophoresis signal after 10 sec. Points measured above 4.7 μM Aurora A were rejected.

### Cell Culture Experiments.

HeLa Kyoto cells were cultured in high-glucose DMEM with GlutaMAX (Life Technologies) media supplemented with 10% fetal calf serum (FCS) in a humidified 5% CO₂ incubator at 37° C. For plasmid transfections, cells were seeded at 80-90% confluence. 4 μg of plasmid DNA in 100 μl OptiMEM and 4 μl of Lipofectamine 2000 (Life Technologies) in 100 μl OptiMEM were incubated in parallel for 5 minutes, mixed for 20 minutes and added to each well. All Aurora A clones were constructed using full-length Aurora A as a template with appropriate PCR primer pairs. The amplified products were subcloned into a pcDNA3-GFP vector (Merdes et al., J Cell Biol, 2000, 149, 851-862). GFP-Aurora A_{R137A} and GFP-Aurora A_{W277A} were engineered using a site directed mutagenesis kit (Agilent Technologies, no. 210515) with appropriate primers. For determining the mitotic index and the chromosome misalignment phenotype, cells were incubated with various concentrations of compounds for 20 h before analysis. Cells were likewise treated with 0.25 μM MLN8237 (Selleckchem, no. S1133) or 5 μM ZM447439 (Selleckchem, no. S1103). For flow cytometry analysis, 50 nM MLN8037 and 2.5 μM ZM447439 were used. For rescue experiments, HeLa Kyoto cells were first transfected with GFP-Aurora A, GFP-Aurora A_{R137A} or GFP-Aurora A_{W277A} for 20 h, followed by incubation with 9 for another 20 h before cells were fixed with cold methanol and stained. To assess the impact of compound 9 on cell cycle progression, DNA content was analyzed after propidium iodide staining using FACS (BD Accuri C6 flow cytometer).

### Indirect Immunofluorescence.

For immunofluorescence, cells were fixed in cold methanol, washed in PBT (PBS supplemented with 0.05% Tween-20) and stained with the following primary antibodies: 1:200 rabbit anti-pT288 (Cell Signaling, C39D8), 1:200 rabbit anti-pH3S10 (Cell Signaling, D2C8), 1:500 mouse anti-α-tubulin (Transduction Laboratories, 612709), 1:300 mouse anti-GFP (MAB3580, Millipore), 1:200 anti-TPX2 (Santa Cruz Biotechnology, sc-32863), anti-Aurora A (Invitrogen, 458900). Secondary antibodies were anti-mouse conjugated to Alexa488 or Alexa568, as well as anti-rabbit conjugated to Alexa488 or Alexa568, all used at 1:500 (Invitrogen). Confocal images were acquired on a Zeiss LSM 710 confocal microscope equipped with a Axiocam MRm (B/VVW) CCD camera using a 63×NA 1.0 oil objective and processed in ImageJ and Adobe Photoshop, maintaining relative image intensities within a series.

### Intrinsic Tryptophan Fluorescence.

Binding experiments were performed by mixing Aurora A (500 nM, 375 nM, and 150 nM for 77, 9, and 88, respectively) with varying concentrations (0-100 μM) of the different compounds in buffer (final DMSO concentration 1%), followed by incubation for 15 min at room temperature. Tryptophan fluorescence spectra were recorded at room temperature using an Infinite M1000 Pro plate reader (Tecan) and Corning 96 well half area, flat bottom, non-binding surface, black polystyrene plates. Excitation occurred at 284 nm (bandwidth ±5 nm) and emission at 340 nm (bandwidth ±10 nm). The gain was calculated from the well with the highest protein concentration. Each measurement was done in triplicate.

1. A compound comprising the general formula (1a) or (1b), in particular (1a), 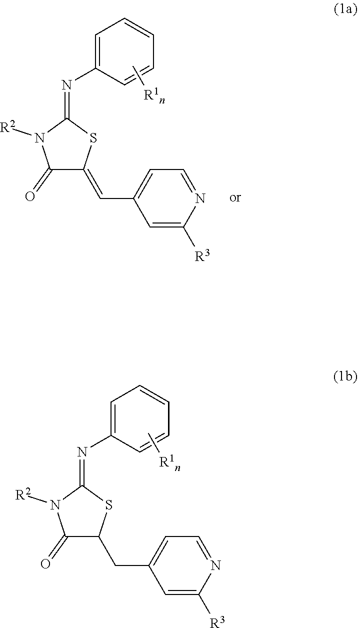
wherein
R¹ is selected from —I, —Br, —Cl, —F, C₁-C₆-alkyl, —O(CH₂)ₘCH₃, —(CH₂)ₘOCH₃, C₁-C₆-haloalkyl, a substituted or unsubstituted cycloalkyl, a substituted or unsubstituted heterocycle, a substituted or unsubstituted aryl or a substituted or unsubstituted heteroaryl, wherein
n is 0, 1, 2, 3, 4 or 5 and
m is 0, 1, 2, 3, 4 or 5
R² is selected from H, C₁-C₆-alkyl, C₁-C₆-haloalkyl, C₃-C₆-cycloalkyl, —CH₂—(C₃-C₆-cycloalkyl), or —(CH₂)ᵣOCH₃, wherein
r is 1, 2, 3, 4 or 5
R³ is
—NH₂, —NH—R⁴, —NHC(═O)—R⁴, —NHC(═O)NH—R⁴, —NHC(═S)—R⁴ or —NHC(═S)NH—R⁴, wherein
R⁴ is selected from
a substituted or unsubstituted cycloalkyl, a substituted or unsubstituted heterocycle, a substituted or unsubstituted aryl or a substituted or unsubstituted heteroaryl, or
-L-R⁵, wherein
L is selected from
C₁-C₅-alkyl,
a substituted or unsubstituted cycloalkyl, a substituted or unsubstituted heterocycle, a substituted or unsubstituted aryl, or a substituted or unsubstituted heteroaryl, and
R⁵ is selected from —OH, —CH₂OH, —NH₂, —COOH, —CONH₂, —CONH—R⁶ or carboxylic acid isosteres, wherein
R⁶ is selected from C₁-C₄-alkyl, in particular C₁-C₂-alkyl.

2. The compound according to claim 1, wherein R¹ is selected from C₁-C₆-alkyl, —I, —Br, —Cl, —F, —O(CH₂)ₘCH₃, —(CH₂)ₘOCH₃, cycloalkyl, in particular C₃-C₆-cycloalkyl, more particularly C₆-cycloalkyl, or C₁-C₆-haloalkyl, in particular R¹ is selected from C₁-C₆-alkyl, —O(CH₂)ₘCH₃, —I, —Br, —Cl, —F or C₁-C₆-haloalkyl, wherein m is 0, 1, 2, 3, 4 or 5.

3. The compound according to claim 1, wherein R¹ is selected from C₁-C₄-alkyl, in particular C₁-C₃-alkyl, more particularly methyl, ethyl or isopropyl, C₁-C₄-haloalkyl, in particular —CH2CF3, —CHFCF3, —CF2CF3, —CHF2, —CH2F or —CF₃, more particularly —CH₂CF₃ or CF₃, —O(CH₂)ₘCH₃, —I, —Br, —Cl, or —F, in particular R¹ is selected from —Br, —CF₃ or ethyl, more particularly R¹ is ethyl, wherein m is 0, 1, 2 or 3.

4. The compound according to claim 1, wherein n of R¹ₙ is 0, 1 or 2, in particular n is 1, wherein more particularly R¹ is a para-substitution.

5. The compound according to claim 1, wherein R² is selected from H, C₁-C₆-alkyl, or —(CH₂)ᵣOCH₃, in particular H, C₁-C₄-alkyl or —(CH₂)ᵣOCH₃, with r being 1, 2, 3, 4 or 5 wherein in particular r is 1, 2 or 3, more particularly r is 1 or 2.

6. The compound according to claim 1, wherein R² is selected from H, C₁-C₂-alkyl or —(CH₂)₂OCH₃, in particular R² is C₁-C₂-alkyl, more particularly R² is —CH₃.

7. The compound according to claim 1, wherein R³ is selected from —NH₂, —NH—R⁴ or —NHC(═O)—R⁴, in particular from —NH—R⁴ or —NHC(═O)—R⁴.

8. The compound according to claim 1, wherein R⁴ is selected from a substituted or unsubstituted aryl, in particular substituted or unsubstituted C₆-aryl, more particularly phenyl, or a substituted or unsubstituted heteroaryl, in particular substituted or unsubstituted C₆-heteroaryl, more particularly 2-pyridyl, or -L-R⁵, wherein in particular R⁴ is -L-R⁵.

9. The compound according to claim 1, wherein L is selected from C₁-C₅-alkyl, in particular C₁-C₃-alkyl, a substituted or unsubstituted aryl, or a substituted or unsubstituted heteroaryl, in particular L is selected from C₆-aryl or C₆-heteroaryl, in particular pyridyl, or C₁-C₅-alkyl, in particular C₁-C₃-alkyl.

10. The compound according to claim 1, wherein R⁵ is selected from —CH₂OH, —NH₂, —COOH, —CONH₂, —CONH—R⁶, tetrazole, in particular —CH₂OH, —NH₂ or —COOH, more particularly —COOH, with R⁶ being selected from C₁-C₄-alkyl, in particular C₁-C₂-alkyl.

11. The compound according to claim 1, wherein
R³ is —NHC(═O)-L-R⁵, wherein L is selected from C₁-C₃-alkyl, and R⁵ is selected from —COOH or —NH₂, in particular —COOH,
or wherein
R³ is —NH-L-R⁵, wherein L is a substituted or unsubstituted aryl, in particular a 6-membered substituted or unsubstituted aryl, more particularly phenyl, or a substituted or unsubstituted heteroaryl, in particular a 6-membered substituted or unsubstituted heteroaryl, more particularly 2-pyridyl, and R⁵ is selected from —COOH or —NH₂, in particular —COOH.

12. A compound according to claim 1 for use as a medicament.

13. A compound according to claim 1 for use in the treatment of cancer.

14. A compound according to claim 1 for use as an inhibitor of Aurora A.

15. A method for treating or preventing a disease, comprising administrating a compound according to any one of claim 1 to a patient in need thereof, in particular in a pharmaceutically effective amount, more particularly wherein said disease is cancer.
